(19) United States
(12) Patent Application Publication
(10) Pub. No.: US 2026/0024196 A1
(43) Pub. Date: Jan. 22, 2026

(54) SYSTEM AND METHOD FOR IMPROVED MR IMAGING OF BRAIN VENTRICLES

(71) Applicant: Aspect Imaging Ltd., Shoham (IL)

(72) Inventor: Gil Farkash, Shoham (IL)

(73) Assignee: Aspect Imaging Ltd., Shoham (IL)

(21) Appl. No.: 18/780,239

(22) Filed: Jul. 22, 2024

(51) Int. Cl. G06T 7/00; A61B 5/055; G06T 7/13; G06T 7/62; G16H 50/20

(52) U.S. Cl. CPC G06T 7/0012; A61B 5/055; G06T 7/13; G06T 7/62; G16H 50/20; G06T 2207/10088; G06T 2207/20084; G06T 2207/30016

(57) ABSTRACT

An artificial intelligence (AI) engine is trained on a plurality of annotated magnetic resonance (MR) images of a patient's brain. A plurality of MR images of a patient's head is provided. For each MR image in the plurality of provided MR images, the AI engine detects a plurality of edges of the brain and determine a biparietal diameter (BP) value, detects a plurality of frontal horn edges, and detects a plurality of occipital horn edges. A correction module determines that at least one detected edge is associated with a non-ventricular body and updates the edge to correspond to the applicable horn. The AI engine determines a frontal horn diameter (F) value, and a occipital horn diameter (O) value. An indication module provides an indication on abnormal dilation of the patient's brain ventricles based on a maximum F value, a maximum O value, and the maximum BP value.

### BACKGROUND

The disclosure relates generally to magnetic resonance (MR) imaging.

The ventricular system includes four interconnected cavities known as brain or cerebral ventricles. The ventricles are filled with cerebrospinal fluid or CSF that is circulated through the ventricles and the continuous central canal of the spinal cord. The four ventricles include two lateral ventricles (left and right, one for each hemisphere), a third ventricle and a fourth ventricle. Each of the left and right lateral ventricles resembles a c-shaped cavity that begins at an inferior horn in the temporal lobe, travels through a body in the parietal lobe and frontal lobe, and ultimately terminates at the interventricular foramina where each lateral ventricle connects to the central third ventricle. Along this path, a posterior horn extends into the occipital lobe and an anterior horn extends into the frontal lobe. The posterior horn is also known as the occipital horn and the anterior horn is also known as the frontal lobe.

A parameter known as the frontal and occipital horn ratio (FOHR) is the measurement of the average of the frontal horn diameter (F) and occipital horn diameter (O) divided by the biparietal diameter (BP) as per the following equation:

$$FOHR = \frac{[ \frac{F + O}{2} ]}{BP} ,$$

where the frontal horn diameter F is the horizontal distance between the widest edges of the frontal horn (i.e., the length a horizontal line defined by the outermost edge of the left frontal horn and the outermost edge of the right frontal horn); the occipital horn diameter O is the horizontal distance between edges of the occipital horn (i.e., the length of a horizontal line defined by the outermost edge of the left occipital horn and the outermost edge of the right occipital horn), and the biparietal diameter BP is the horizontal distance between the widest edges of the cortex.

The FOHR gives an indication of an abnormal dilation of the brain ventricles, related to intraventricular hemorrhage (IVH) or post-hemorrhagic ventricular dilation (PHVD). It may be clinically used to determine the appropriate time for intervention. For example, for neonates having a FOHR greater than 0.5 may be referred to shunt surgery.

Identifying the edges of the front horn and the occipital horn on MR images can be challenging. Similarly challenging is identifying the frontal horn diameter (including the lateral edges of the left and right frontal horns that define the frontal horn diameter), the occipital horn diameter (including the lateral edges of the left and right occipital horns that define the occipital horn diameter), and the biparietal diameter (including the lateral edges of the cortex that define the biparietal diameter). Another technical problem associated with the art is the inability of MR systems to distinguish between ventricles on the one hand, and non-ventricle bodies on the other hand in MR images. For example, a fluid-filled cyst located adjacent to a ventricle, itself filled with CSF, may be indistinguishable to the ventricle.

Accordingly, a need exists to accurately identify on MR images the inputs necessary to determine the FOHR parameter. That need requires the ability to accurately distinguish, using imaging technology, a ventricle body from a non-ventricle body.

### BRIEF DESCRIPTION OF THE DRAWINGS

The accompanying drawings illustrate several embodiments, and together with the description, serve to explain the disclosed principles. One skilled in the relevant art will understand, however, that embodiments can be practiced without all of the specific details of the illustrated examples. Likewise, one skilled in the relevant art will also understand that the technology may include well-known structures or functions not specifically illustrated to avoid unnecessarily obscuring the relevant descriptions of the various examples. In the drawings:

FIG. 1 illustrates an exemplary schematic of a system for improved MR imaging of brain ventricles in accordance with some embodiments of the disclosure.

FIG. 2 illustrates an axial MR slice of a brain annotated to indicate a biparietal diameter, a frontal horn diameter, and a occipital horn diameter as may be used to train the AI engine associated with FIG. 1, in accordance with some embodiments of the disclosure.

FIG. 3 illustrates a coronal MR slice of a brain annotated to indicate a biparictal diameter, a frontal horn diameter, and a occipital horn diameter as may be used to train the AI engine associated with FIG. 1, in accordance with some embodiments of the disclosure.

FIG. 4 illustrates an axial MR slice of a brain annotated to indicate the location of the anterior commissure in the image.

FIG. 5 illustrates a coronal MR slice of a brain annotated to indicate the location of the anterior commissure in the image.

FIG. 6 illustrates an axial MR slice of a brain annotated to indicate the location of the posterior commissure in the image.

FIG. 7 illustrates a coronal MR slice of a brain annotated to indicate the location of the posterior commissure in the image.

FIG. 8 illustrates an exemplary method for improved MR imaging of brain ventricles in accordance with some embodiments of the disclosure.

### DETAILED DESCRIPTION

Several embodiments are discussed below in more detail in reference to the figures, where common numerals refer to the same method block, feature or component. Other embodiments in addition to those described herein are within the scope of the disclosure. Moreover, a person of ordinary skill in the art will understand that embodiments of the disclosure may have configurations, components, and/or procedures in addition to those shown or described herein and that these and other embodiments may be implemented without several of the configurations, components, and/or procedures shown or described herein without deviating from the disclosure. Reference throughout this description to “one embodiment,” “an embodiment,” “one or more embodiments,” an “nth embodiment,” or “some embodiments” means that a particular feature, support structure or characteristic described in connection with the embodiment is included in at least one embodiment of the present disclosure. Thus, use of such terminology is not necessarily referring to the same embodiment. For example, it is expressly contemplated that the features described herein may be combined in any suitable manner in one or more embodiments.

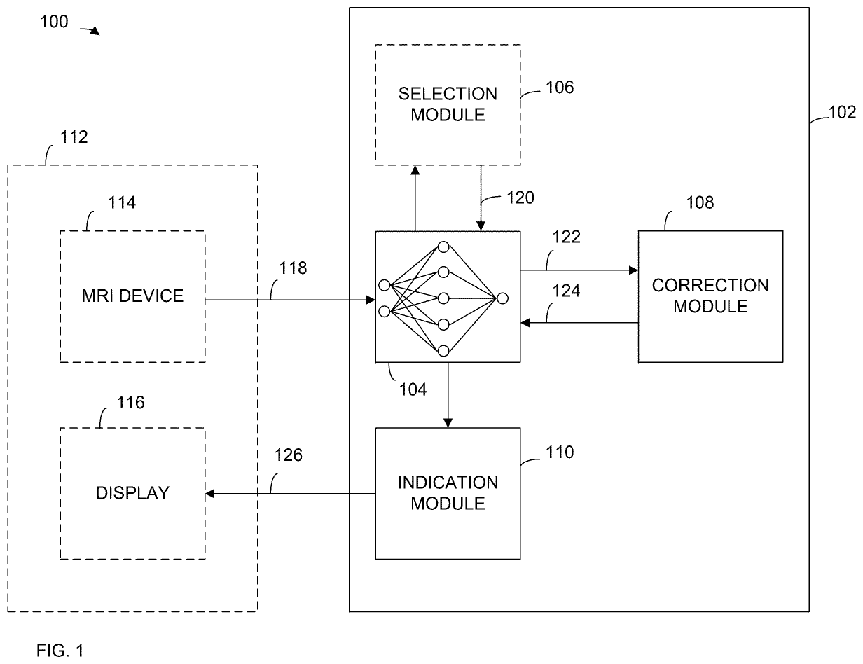

With reference to FIG. 1, an exemplary schematic 100 of system 102 for improved MR imaging of brain ventricles is disclosed. System 102 may include trained artificial intelligence (AI) engine 104, correction module 108, and indication module 110. System 102 may optionally include selection module 106 and imaging subsystem 112, which may include a magnetic resonance (MR) device 114 and a display 116.

AI engine 104 may be one or more convolutional neural networks. In some embodiments, AI engine 104 is one or more convolutional regression neural networks. In other embodiments, AI engine 104 is one or more convolutional segmentation neural networks. In yet other embodiments, AI engine 104 is a plurality of convolutional neural networks including both convolutional regression neural networks and convolutional segmentation neural networks. AI engine 104 may be trained to process 2D and/or 3D MR images. AI engine 104 may be trained on a plurality of annotated MR images. The annotated images may be 2D and/or 3D MR images. The annotated MR images may correspond to unique two dimensional MR slices of a brain of the same or different persons. The two dimensional MR slices may include a T1-weighted axial slice, a T2-weighted axial slice, a T1-weighted coronal slice, and/or a T2-weighted coronal slice. In some embodiments, the same type of slice is used to train AI engine 104. For example, all of the MR slices may be T2-weighted axial slices. In other embodiments, various types of slices may be used to train AI engine 104. For example, some MR slices may be T2-weighted axial slices and other MR slices may be T1-weighted coronal slices. The annotated MR images may correspond to unique three dimensional MR slices of a brain. The annotated MR images may depict frontal and occipital horns. The annotated MR images may be annotated to identify or indicate those edge portions of the frontal horn that correspond to the frontal diameter F, those edge portions of the occipital horn that correspond to the occipital diameter O and those edge portions of the cortex that correspond to the biparietal diameter BP. The annotations may include lines that connect the respective pairs of edges. For example, the annotations may include a line that identifies the frontal diameter F, the occipital diameter O and the biparietal diameter BP.

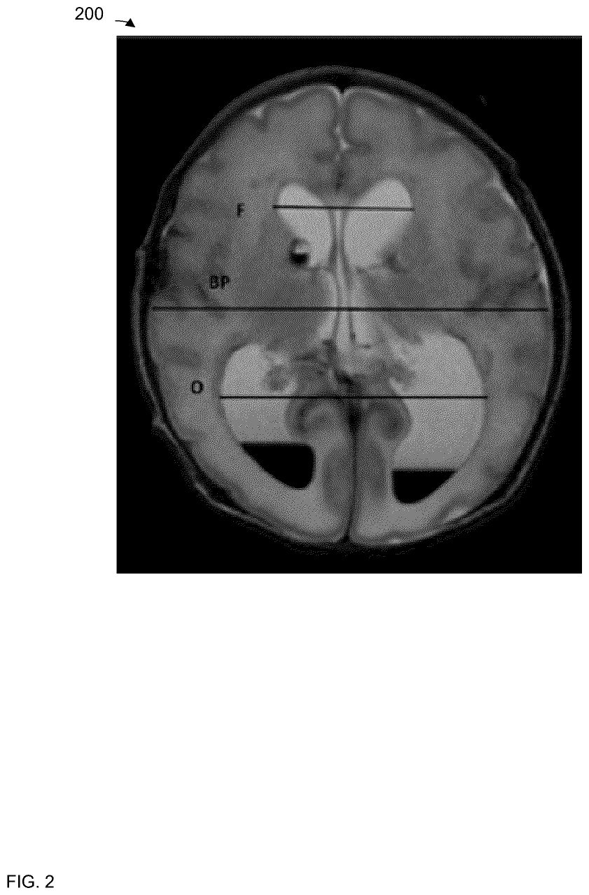

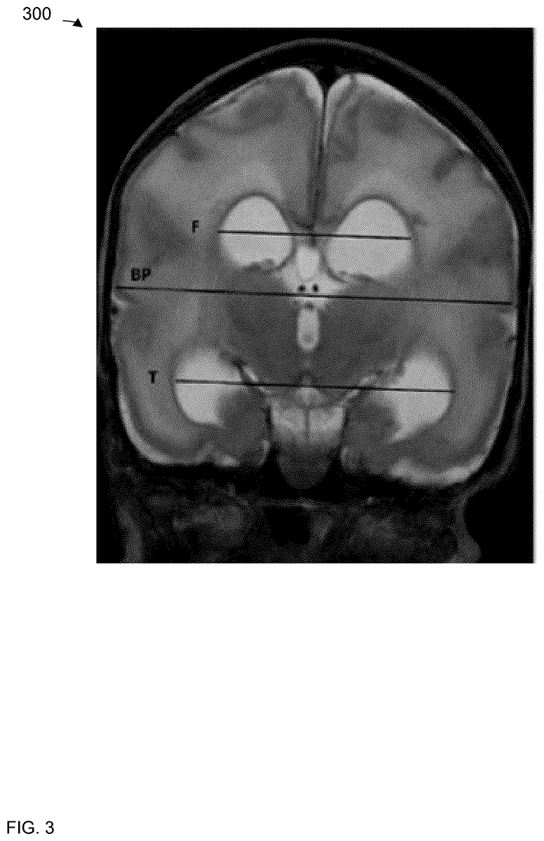

FIG. 2 illustrates an axial MR slice 200 of a brain annotated to indicate a biparietal diameter BP, a frontal horn diameter F, and a occipital horn diameter O as may be used to train the AI engine 104 of FIG. 1. FIG. 3 illustrates a coronal MR slice 300 of a brain annotated to indicate a biparietal diameter BP, a frontal horn diameter F, and a occipital horn diameter O as may be used to train the AI engine 104 of FIG. 1.

In some embodiments, the annotated MR images may include annotations identifying the anterior commissure and the posterior commissure. In other embodiments, the annotated MR images may include annotations that identify the anterior commissure-posterior commissure line (AC-PC line). The AC-PC line may be the line passing through the superior edge of the anterior commissure and the inferior edge of the posterior commissure. Alternatively, the AC-PC line may be the line through the midpoint of both the anterior commissure and posterior commissure.

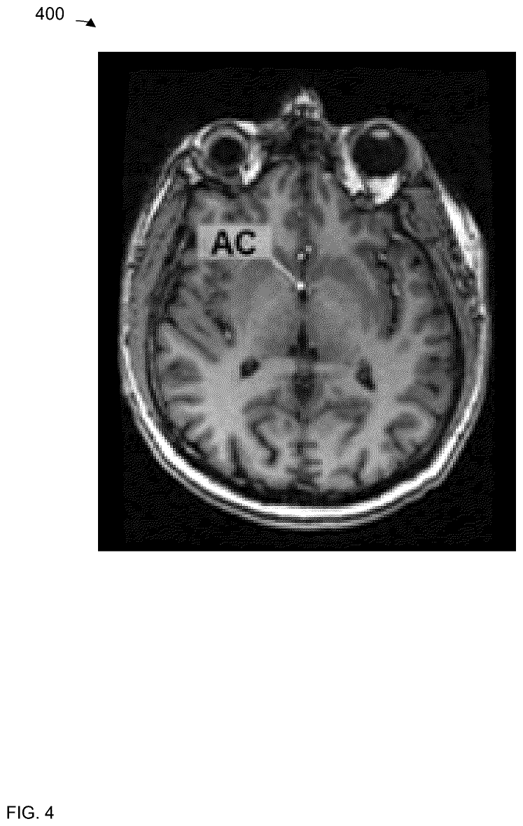

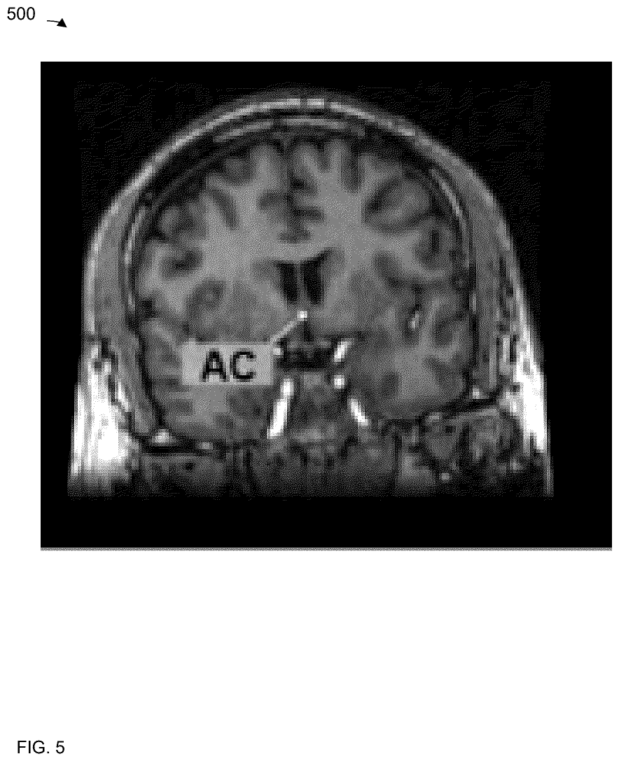

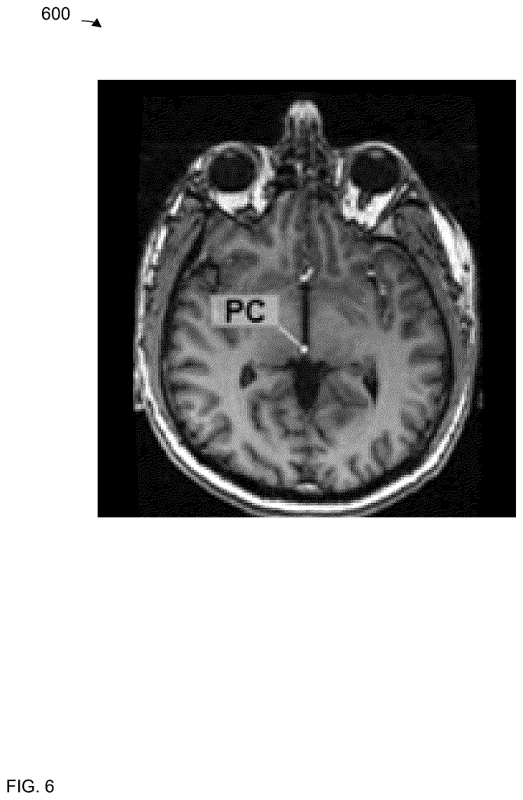

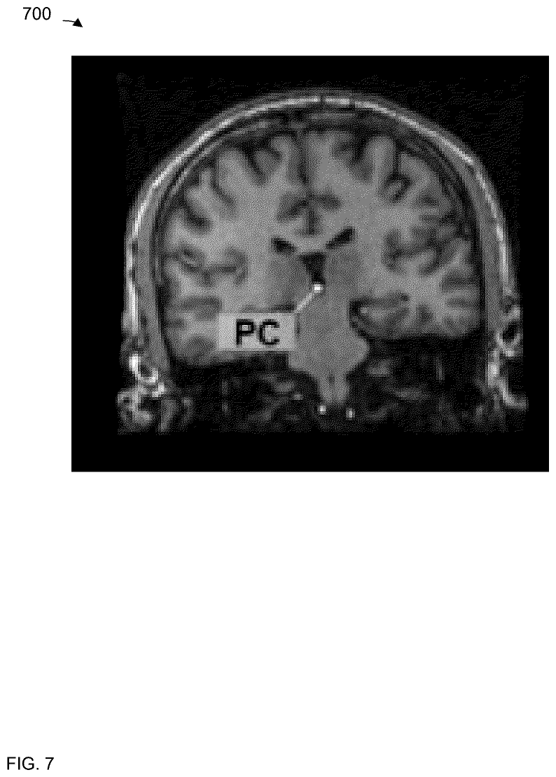

FIG. 4 illustrates an axial MR slice 400 of a brain annotated to indicate the location of the anterior commissure AC. FIG. 5 illustrates a coronal MR slice 500 of a brain annotated to indicate the location of the anterior commissure AC. FIG. 6 illustrates an axial MR slice 600 of a brain annotated to indicate the location of the posterior commissure PC. FIG. 7 illustrates a coronal MR slice 700 of a brain annotated to indicate the location of the posterior commissure PC.

Using the plurality of annotated MR images, the trained AI engine 104 may be trained to identify the boundaries of the frontal horns, the occipital horns, the outermost lateral edges of the frontal horns (e.g., the left and right frontal horns), the outermost lateral edges of the occipital horns (e.g., the left and right occipital horns), and the outermost lateral edge of the cortex in MR images, e.g., in axial and/or coronal MR images. In some embodiments, the AI engine 104 may be trained to identify the AC and the PC and the AC-PC line. In some embodiments, the AI engine 104 may be trained to identify an orientation of the AC-PC line. In some embodiments, the AI engine 104 may be trained to identify the distance between one or more pairs of lateral edges associated with the frontal horns (e.g., left and right frontal horns), the distance between one or more pairs of lateral edges associated with the occipital horns (e.g., left and right occipital horns), and the distance between one or more pairs of lateral edges of the cortex. In certain embodiments, these distances may be along a horizontal axis defined with reference to the orientation of the AC-PC line. In some embodiments, the horizontal axis may be orthogonal or sufficiently orthogonal (e.g., within x number of degrees, where x is 5, 10, 15, or 20) to the orientation of the AC-PC line. For example, the AI engine 104 may be trained to identify a frontal horn diameter by determining the distance between the widest edges of the frontal horns along a horizontal axis defined based on the orientation of the AC-PC line. Similarly, the AI engine 104 may be trained to identify a occipital horn diameter by determining the distance between the widest edges of the occipital horns along a horizontal axis defined based on the orientation of the AC-PC line. Likewise, the AI engine 104 may be trained to identify a biparietal diameter by determining the distance between the widest edges of the cortex along a horizontal axis defined based on the orientation of the AC-PC line.

Returning to FIG. 1, system 102 and in particular AI engine 104 may receive a first plurality of MR images 118 of a patient's head. Each MR image in the first plurality of MR images 118 may correspond to a unique two dimensional MR slice that is one of a T1-weighted axial slice, a T2-weighted axial slice, a T1-weighted coronal slice, and a T2-weighted coronal slice. In other embodiments, each MR image in the first plurality of MR images 118 may correspond to a unique three dimensional MR slice. In one embodiment, as is depicted in exemplary FIG. 1, first plurality of MR images 118 may be transmitted by imaging subsystem 112 and in particular MR device 114. In such embodiments, each MR image in the second plurality of MR images 118 may corresponds to a unique two dimensional MR slice that is one of a T1-weighted axial slice, a T2-weighted axial slice, a T1-weighted coronal slice, and a T2-weighted coronal slice. MR device 114 may be the Embrace neonatal MRI system manufactured by Aspect Imaging, Ltd. or any suitable magnetic resonance imaging device that is capable of generating such MR images.

For each MR image of the first plurality of MR images 118, the trained AI engine 103 may detect a plurality of edges of the cortex and determine a biparietal diameter (BP) value. In one embodiment, the trained AI engine 103 may determine an orientation of the anterior commissure—posterior commissure (AC-PC) midline on each MR image of a first plurality of MR images 118 and use such orientation to determine the BP value for each such image, where the BP value is the distance between the widest edges of the cortex along a horizontal axis defined based on the orientation of the AC-PC line. In some embodiments, the horizontal axis may be orthogonal or sufficiently orthogonal (e.g., within x number of degrees, where x is 5, 10, 15, or 20) to the orientation of the AC-PC line.

In some embodiments, the MR image having the maximum BP value is identified (e.g., by selection module 106). One or more other MR images of the first plurality of MR images 118 are also identified and/or selected (e.g., by selection module 106) based on the identified MR image with the maximum BP value. In some embodiments, the one or more other MR images of the first plurality of MR images 118 may be identified and/or selected based on their slice location relative to the slice corresponding to the MR image with the maximum BP value. For example, the one or more other MR images of the first plurality of MR images 118 may be correspond to a MR slice within n number of slices of the MR slice associated with the MR image with the maximum BP value, where n is 5, 10, 15, 20, 25, 30, 35, 40, 45, and 50. Put another way, the one or more other MR images may be within the range of +3/−3 slices, or within the range of +3/−2 slices, or within the range of +2/−3 slices, etc. Collectively, the MR image with the maximum BP value and the one or more other MR images of the first plurality of MR images 118 so identified and/or selected may constitute a second plurality of MR images 120. Although depicted as a separate module, selection module 106 may be incorporated within AI engine 104.

For each MR image of the second plurality of MR images 120, the AI engine 104 may be configured to further detect a plurality of frontal horn edges (e.g., the lateral-most edges) and a plurality of occipital horn edges (e.g., the lateral-most edges). Collectively, these pluralities of detected edges 122 may be passed to a correction module 108 that is configured to determine whether at least one detected edge of the pluralities of detected edges 122 is associated with a non-ventricular body (e.g., a cyst or other mass that is indistinguishable or sufficiently indistinguishable from a ventricular horn).

In some embodiments, correction module 108 may be configured to make such a determination based on the AC-PC midline. In such embodiments, to the extent the AC-PC midline is not already determined for each MR image undergoing such analysis, the AI engine 104 may be configured to identify the AC-PC midline on each such MR image, as was generally described above. Correction module 108 may receive at least one pair of horn edges that are along a horizontal axis defined with reference to the orientation of the AC-PC line. In some embodiments, the horizontal axis may be orthogonal or sufficiently orthogonal (e.g., within x number of degrees, where x is 5, 10, 15, or 20) to the orientation of the AC-PC line. Correction module 108 may be configured to determine whether the line defined by each pair of horn edges is symmetric about the AC-PC midline. In other words, whether the distance from the edge associated with a left horn to the AC-PC midline is the same or substantially the same (e.g., within n percent, where n is 5, 10, 15, or 20) as the distance from the edge associated with a right horn to the AC-PC midline. If the distances are not the same or substantially the same then correction module 108 may determine that the edge with the longer distance is associated with a non-ventricular body.

In other embodiments, correction module 108 may be configured to make such a determination based on smoothness of the plurality of edges themselves. In such an embodiment, correction module 108 may be configured to determine the contours defined by the plurality of edges are sufficiently differentiable. Other techniques may be used to determine the degree to which the plurality of edges are sufficiently smooth. Any deviations or any material deviations from smoothness may indicate the presence of a non-ventricular body.

Correction module 108 may be configured to update or correct any edges that are associated with a non-ventricular body. For example, if correction module 108 identified a non-ventricular body using the symmetry analysis, the updated edge value would be the value that would make the left and right distances to the AC-PC midline the same or substantially the same. Likewise, if correction module 108 identified a non-ventricular body using a smoothness analysis, the updated edge value would be the value that would make y neighboring edges in the plurality of edges smooth or sufficiently smooth, where y is 5, 10, 15, 20, 25, 30, 35, 40, 45, or 50. Correction module 108 may return the updated edge values 124 to AI engine 104.

For each MR image of the second plurality of MR images 120, the trained AI engine 104 may be configured to determine a frontal horn diameter (F) value based on the plurality of detected frontal horn edges and determine a occipital horn diameter (O) based on the plurality of detected occipital horn edges. In one embodiment, at least one edge in the pluralities of detected horn edges was updated by correction module 108. The F and O values may be the distance between the widest lateral edges of respective horns (e.g., the widest lateral edges of the frontal horns or the widest lateral edges of the occipital horns) along a horizontal axis defined with reference to the orientation of the AC-PC line. In some embodiments, the horizontal axis may be orthogonal or sufficiently orthogonal (e.g., within x number of degrees, where x is 5, 10, 15, or 20) to the orientation of the AC-PC line (as may be determined by the AI engine 104).

Indication module 110 may be configured to determine a frontal and occipital horn ratio (FOHR) based on at least one maximum F value, at least one maximum O value, and at least one maximum BP value among such F, O and BP values for the second plurality of MR images 120. In one embodiment, AI engine 104 and/or indication module 110 may determine such maximal F, O and BP values. The FOHR may be determined by taking the average of the maximum F and maximum O values and dividing that result by the maximum BP value. If the result is greater than a predetermined value (e.g., 0.5), the indication module 110 may provide indication on abnormal dilation of the patient's brain ventricles.

Indication module 110 may further generated annotated MR image data 126. Annotated MR image data 126 may include an MR image (e.g., an MR image from the second plurality of MR images 120) annotated to include (a) horn edges associated with the maximum F value; (b) a line defined by the horn edges associated with the maximum F value; (c) horn edges associated with the maximum O value; (d) a line defined by the horn edges associated with the maximum O value; (c) brain edges associated with the maximum BP value; (f) a line defined by the brain edges associated with the maximum BP value; (g) the FOHR; and (h) the indication result (e.g., a likelihood of abnormal dilation). In one embodiment, annotated MR image data 126 may be displayed on a suitable display (e.g., display 116) associated with imaging subsystem 112.

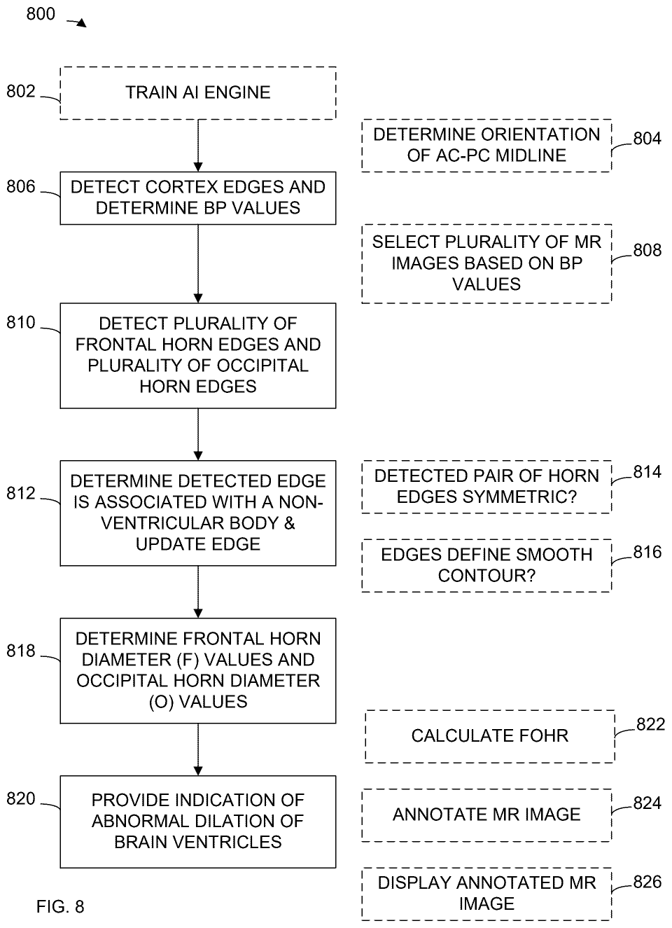

FIG. 8 illustrates an exemplary method 800 for improved MR imaging of brain ventricles in accordance with some embodiments of the disclosure. Each of the method blocks may be implemented using the hardware, engines, and modules described in connection with FIG. 1. Method 800 may optionally include training an AI engine at block 802. The method 800 may continue with block 806 where trained engine may detect cortex edges and determine biparictal diameter values for a plurality of MR images. Optionally, method 800 may include determining the orientation of an AC-PC midline at block 804. The determination of the BP values (per block 806) may be based on such orientation.

Method 800 may further include block 808 where a plurality of MR images may be selected based on the determined BP values. In block 810, a plurality of frontal horn edges (e.g., lateral edges) and a plurality of occipital horn edges (e.g., lateral edges) may be detected.

In method block 812, a determination is made as to whether any detected edges are associated with a non-ventricular body. The determination may be made by determining whether edge pairs are symmetric about the AC-PC midline (optional block 814) or by determining whether the edges define a smooth contour (optional block 816). If a determination is made that a detected edge is associated with a non-ventricular body, the edge is updated in block 812.

At block 818 frontal and occipital horn diameters are obtained. And at block 820 an indication of abnormal dilation of brain ventricles is provided based on the diameters. Optionally, method 800 may include calculating the FOHR (block 822), annotating an MR image with horn diameters, FOHR, and the indication (block 824), and displaying the annotated MR image (block 826).

The technology and techniques provide technical solutions to technical problems associated with accurately using MR technology to identify frontal and occipital horns and cortex boundaries in MR slices, while critically distinguishing between ventricles on the one hand, and non-ventricle bodies on the other hand. The description set forth herein improves the field of MR imaging at least insofar as the description takes steps to improve MR imaging by extracting from F and O values data associated with non-ventricle bodies and then accurately displaying information that is correctly associated only with the frontal horn diameter, the occipital horn diameter, and the biparietal diameter. These improvements are practical and unconventional and improve the functioning of MR and related imaging systems. The technology and techniques described herein can be applied to any MRI system, including but not limited to MRI systems employing super-conducting magnets and permanent magnets, and to both 2D and 3D MRI.

As used herein, the terms “module,” “logic,” “engine” and its and their components may refer to any single or collection of circuit(s), integrated circuit(s), hardware processor(s), processing device(s), transistor(s), non-transitory memory(s), storage devices(s), non-transitory computer readable medium(s), combination logic circuit(s), or any combination of the above that is capable of providing a desired operation(s) or function(s). For example, a “module”, “logic” or “engine” may take the form of a hardware processor executing instructions from one or more non-transitory memories, storage devices, or non-transitory computer readable media, or a dedicated integrated circuit. “Non-transitory memory,” “non-transitory computer-readable media,” and “storage device” may refer to any suitable internal or external non-transitory, volatile or non-volatile, memory device, memory chip(s), or storage device or chip(s) such as, but not limited to system memory, frame buffer memory, flash memory, random access memory (RAM), read only memory (ROM), a register, a latch, or any combination of the above. A “hardware processor” may refer to one or more dedicated or non-dedicated: hardware micro-processors, hardware micro-controllers, hardware sequencers, hardware micro-sequencers, digital signal hardware processors, hardware processing engines, hardware accelerators, applications specific circuits (ASICs), hardware state machines, programmable logic arrays, any integrated circuit(s), discreet circuit(s), etc. that is/are capable of processing data or information, or any suitable combination(s) thereof. A “processing device” may refer to any number of physical devices that is/are capable of processing (e.g., performing a variety of operations on) information (e.g., information in the form of binary data or carried/represented by any suitable media signal, etc.). For example, a processing device may be a hardware processor capable of executing executable instructions, a desktop computer, a laptop computer, a mobile device, a hand-held device, a server (e.g., a file server, a web server, a program server, or any other server), any other computer, etc. or any combination of the above. An example of a processing device may be a device that includes one or more integrated circuits comprising transistors that are programmed or configured to perform a particular task. “Executable instructions” may refer to software, firmware, programs, instructions or any other suitable instructions or commands capable of being processed by a suitable hardware processor. The terms “adapted to” and “configured to” mean physically adapted and/or configured to.

While illustrative embodiments have been described herein, the scope thereof includes any and all embodiments having equivalent elements, modifications, omissions, combinations (e.g., of aspects across various embodiments), adaptations and/or alterations as would be appreciated by those in the art based on the present disclosure. For example, the number and orientation of components shown in the exemplary systems may be modified.

Those skilled in the art will appreciate that the method blocks need not necessarily be performed in the order in which they are depicted in the figures or described herein. For example, the order of the acts may be rearranged; some acts may be performed in parallel; shown acts may be omitted, or other acts may be included; a shown act may be divided into sub acts, or multiple shown acts may be combined into a single act, etc. Similarly, those skilled in the art will appreciate that one or more components depicted in functional block diagrams may be omitted without deviating from the scope of the disclosure. In particular and without limiting the immediately foregoing sentence, those components denoted in dashed lines may be omitted in one or all embodiments.

It will be appreciated by those skilled in the art that the above-described facility may be straightforwardly adapted or extended in various ways. While the foregoing description makes reference to particular embodiments, the scope of the invention is defined solely by the claims that follow and the elements recited therein.

What is claimed is:

1. A system for improved MR imaging of brain ventricles, the apparatus comprising a trained artificial intelligence (AI) engine trained on a plurality of annotated MR images of a patient's brain, a correction module, and an indication module, wherein:
for each MR image of a first plurality of MR images of a patient's head, the trained AI is configured to:
detect a plurality of edges of the cortex and determine a biparietal diameter (BP) value,
detect a plurality of frontal horn edges, and
detect a plurality of occipital horn edges,
a correction module configured to determine at least one detected edge of the pluralities of detected edges is associated with a non-ventricular body and update said edge to correspond to the applicable horn;
for each MR image of the first plurality of MR images of a patient's head, the trained AI engine is further configured to:
determine a frontal horn diameter (F) value based on the plurality of detected frontal horn edges;
determine a occipital horn diameter (O) value based on the plurality of detected occipital horn edges,
wherein at least one edge in the pluralities of detected horn edges was updated by the correction module; and
the indication module is configured to provide an indication on abnormal dilation of the patient's brain ventricles based on at least one F value, at least one O value, and at least one BP value.

2. The apparatus of claim 1, wherein the trained AI engine is further configured to determine an orientation of the anterior commissure-posterior commissure (AC-PC) midline on each MR image of a first plurality of MR images, and wherein each of the BP value, the F value, and the O value are based on the orientation of the AC-PC midline.

3. The apparatus of claim 1, wherein:
the first plurality of MR images is a subset of a second plurality of MR images of the patient's head,
the trained AI engine is further configured to determine a BP value for each MR image in the second plurality of MR images, and
the apparatus further comprising a selection module configured to select the first plurality of MR images from the second plurality of MR images based on the BP values of the second plurality of MR images.

4. The apparatus of claim 1, wherein each MR image of the first plurality of MR images corresponds to a unique two dimensional MR slice that is one of a T1-weighted axial slice, a T2-weighted axial slice, a T1-weighted coronal slice, and a T2-weighted coronal slice.

5. The apparatus of claim 3, wherein:
each MR image in the second plurality of MR images corresponds to a unique MR slice, and
the first plurality of MR images includes:
the MR image with the maximum BP value among the second plurality of MR images, and
one or more MR images from the second plurality of MR images that correspond to MR slices within n number of slices of the MR slice associated with the MR image with the maximum BP value.

6. The apparatus of claim 1, wherein:
the first plurality of MR images is a subset of a second plurality of MR images of the patient's head,
each MR image in the second plurality of MR images corresponds to a unique MR slice,
the trained AI engine is further configured to determine a BP value for each MR image in the second plurality of MR images, and
the apparatus further comprises a selection module configured to select the first plurality of MR images from the second plurality of MR images based on the determined BP values of the second plurality of MR images, wherein the first plurality of MR images includes:
the MR image with the maximum BP value among the second plurality of MR images, and
one or more MR images from the second plurality of MR images that correspond to MR slices within n number of slices of the MR slice associated with the MR image with the maximum BP value.

7. The apparatus of claim 1, wherein the trained AI engine comprises a plurality of trained AI engines.

8. The apparatus of claim 1, wherein the trained AI engine is at least one of:
a trained regression convolutional neural network, and
a trained segmentation convolutional neural network.

9. The apparatus of claim 2, wherein the correction module is configured to determine at least one detected edge of the detected pluralities of edges is an edge of non-ventricular body based on the AC-PC midline.

10. The apparatus of claim 9, wherein:
the trained AI engine is further configured to detect at least one pair of frontal horn edges based on the AC-PC midline and at least one pair of occipital horn edges based on the AC-PC midline, and
the correction module is configured to determine whether a line defined by any pair of edges of the detected pairs is symmetric about the AC-PC midline.

11. The apparatus of claim 1, wherein the correction module is configured to determine whether (a) the plurality of frontal horn edges define a smooth contour and (b) the plurality of occipital horn edges define a smooth contour.

12. The apparatus of claim 1, wherein the indication module is further configured to generate annotated MR image data comprising at least one of:
horn edges associated with a maximum F value;
a line defined by the horn edges associated with the maximum F value;
horn edges associated with a maximum O value;
a line defined by the horn edges associated with a maximum O value;
cortex edges associated with a maximum BP value;
a line defined by the brain edges associated with the maximum BP value;
a frontal and occipital horn ratio (FOHR) based on at least one maximum F value, at least one maximum O value, and at least one maximum BP value; and
the indication on abnormal dilation of the patient's brain ventricles.

13. The apparatus of claim 1, wherein the indication module is further configured to determine a frontal and occipital horn ratio (FOHR) based on at least one maximum F value, at least one maximum O value, and at least one maximum BP value, wherein the indication module is configured to provide the indication on abnormal dilation of the patient's brain ventricles based on the determined FOHR.

14. A method for improved MR imaging of brain ventricles using an artificially intelligence (AI) engine trained by a plurality of annotated MR images of a patient's brain, the method comprising:
for each MR image of a first plurality of MR images of a patient's head:
detecting, by the AI engine, a plurality of edges of the cortex and determine a biparietal diameter (BP) value;
detecting, by the AI engine, a plurality of frontal horn edges; and
detecting, by the AI engine, a plurality of occipital horn edges;
determining, by a correction module, at least one detected edge of the pluralities of detected edges is associated with a non-ventricular body and update said edge to correspond to the applicable horn,
for each MR image of the first plurality of MR images of a patient's head:
determining, by the AI engine, a frontal horn diameter (F) value based on the plurality of detected frontal horn edges; and
determining, by the AI engine, a occipital horn diameter (O) value based on the plurality of detected occipital horn edges,
wherein at least one edge in the pluralities of detected horn edges was updated by the correction module; and
providing, by an indication module, an indication on abnormal dilation of the patient's brain ventricles based on at least one F value, at least one O value, and at least one BP value.

15. The method of claim 14, further comprising, determining, by the trained AI engine, an orientation of the anterior commissure-posterior commissure (AC-PC) midline on each MR image of a first plurality of MR images, and wherein each of the BP value, the F value, and the O value are based on the orientation of the AC-PC midline.

16. The method of claim 14, wherein the first plurality of MR images is a subset of a second plurality of MR images of the patient's head, and the method further comprises:
determining, by the trained AI engine, a BP value for each MR image in the second plurality of MR images; and
selecting, by a selection module, the first plurality of MR images from the second plurality of MR images based on the BP values of the second plurality of MR images.

17. The method of claim 14, wherein each MR image of the first plurality of MR images corresponds to a unique two dimensional MR slice that is one of a T1-weighted axial slice, a T2-weighted axial slice, a T1-weighted coronal slice, and a T2-weighted coronal slice.

18. The method of claim 16, wherein:
each MR image in the second plurality of MR images corresponds to a unique MR slice, and
the first plurality of MR images includes:
the MR image with the maximum BP value among the second plurality of MR images, and
one or more MR images from the second plurality of MR images that correspond to MR slices within n number of slices of the MR slice associated with the MR image with the maximum BP value.

19. The method of claim 14, wherein:
the first plurality of MR images is a subset of a second plurality of MR images of the patient's head, and
each MR image in the second plurality of MR images corresponds to a unique MR slice,
the method further comprising:
determining, by the trained AI engine, a BP value for each MR image in the second plurality of MR images, and
selecting, by a selection module, the first plurality of MR images from the second plurality of MR images based on the determined BP values of the second plurality of MR images, wherein the first plurality of MR images includes:
the MR image with the maximum BP value among the second plurality of MR images, and
one or more MR images from the second plurality of MR images that correspond to MR slices within n number of slices of the MR slice associated with the MR image with the maximum BP value.

20. The method of claim 14, wherein the trained AI engine comprises a plurality of trained AI engines.

21. The method of claim 14, wherein the trained AI engine is at least one of:
a trained regression convolutional neural network, and
a trained segmentation convolutional neural network.

22. The method of claim 15, the method further comprising determining, by the correction module, at least one detected edge of the detected pluralities of edges is an edge of non-ventricular body based on the AC-PC midline.

23. The method of claim 22, the method further comprising:
detecting, by the trained AI engine, at least one pair of frontal horn edges based on the AC-PC midline and at least one pair of occipital horn edges based on the AC-PC midline; and
determining, by the correction module, whether a line defined by any pair of edges of the detected pairs is symmetric about the AC-PC midline.

24. The method of claim 14, the method further comprising determining, by the correction module, whether (a) the plurality of frontal horn edges define a smooth contour and (b) the plurality of occipital horn edges define a smooth contour.

25. The method of claim 1, the method further comprising generating annotated MR image data comprising at least one of:
horn edges associated with a maximum F value;
a line defined by the horn edges associated with the maximum F value;
horn edges associated with a maximum O value;
a line defined by the horn edges associated with the maximum O value;
cortex edges associated with a maximum BP value;
a line defined by the brain edges associated with the maximum BP value;
a frontal and occipital horn ratio (FOHR) based on at least one maximum F value, at least one maximum O value, and at least one maximum BP value; and
the indication on abnormal dilation of the patient's brain ventricles.

26. The method of claim 14, the method further comprising:
determining, by the indication module, a frontal and occipital horn ratio (FOHR) based on at least one maximum F value, at least one maximum O value, and at least one maximum BP value; and
determining, by the indication module, the indication on abnormal dilation of the patient's brain ventricles based on the determined FOHR.
